(19) United States
(12) Patent Application Publication
(10) Pub. No.: US 2022/0110847 A1
(43) Pub. Date: Apr. 14, 2022

(54) USE OF COOLING MATERIALS FOR THE REDUCTION OR INHIBITION OF SALTINESS IN ORALLY ADMINISTERED, IMBIBED OR INGESTED CONSUMER PRODUCTS

(71) Applicant: TAKASAGO INTERNATIONAL CORPORATION, Tokyo (JP)

(72) Inventor: John D. Zanone, Towaco, NJ (US)

(73) Assignee: TAKASAGO INTERNATIONAL CORPORATION, Tokyo (JP)

(21) Appl. No.: 17/423,793

(22) PCT Filed: Jan. 17, 2020
(86) PCT No.: PCT/US2020/014216

§ 371 Date: Jul. 16, 2021

Related U.S. Application Data
(60) Provisional application No. 62/793,749, filed on Jan. 17, 2019.

(51) Int. Cl. A61K 8/42; A61K 8/37; A61K 8/34; A61K 8/35; A61K 8/9789; A61K 8/19; A61Q 11/00

(52) U.S. Cl. CPC A61K 8/42; A61K 8/37; A61K 8/34; A61K 8/345; A61K 2800/92; A61K 8/9789; A61K 8/19; A61Q 11/00; A61K 8/35

(57) ABSTRACT

An orally administered, imbibed, or ingested consumer product, which can ordinarily impart a salty taste and/or after taste to the mouth due to the presence of one or more salts, wherein the salty taste and/or aftertaste is reduced or inhibited by the presence of one or more cooling materials. Related methods of reducing or inhibiting the salty taste and/or aftertaste ordinarily imparted by an orally administered, imbibed, or ingested consumer product to the mouth of the end user are also described.

### CROSS-REFERENCED TO RELATED APPLICATION

This application claims priority to U.S. Provisional Application No. 62/793,749, filed on Jan. 17, 2019, the contents of which are herein incorporated by reference in its entirety.

### FIELD OF THE INVENTION

The present disclosure relates to orally administered, imbibed, or ingested consumer products that ordinarily impart a salty taste and/or after taste to the mouth of the end user, such as those consumer products comprising a high salt content, wherein the salty taste and/or aftertaste is reduced or inhibited by the presence of one or more cooling materials. More particularly, the present disclosure relates to consumer products that can typically have a salty taste and/or aftertaste comprising one or more cooling materials in an amount effective to reduce or inhibit such ordinarily imparted salty taste and/or aftertaste. The consumer product can be an oral care composition such as, by non-limiting example, a baking soda type toothpaste or tooth powder.

### BACKGROUND OF THE INVENTION

Certain consumer products require a high salt content in order to achieve non-flavor qualities or effectiveness, for example, pharmaceutical products having one or more salts as therapeutically active components, or a food product where salt is utilized as a preservative.

More particularly, it is well known that certain oral care compositions (e.g., toothpastes or rinses), contain salt(s) for their abrasive qualities and/or technical effectiveness. However, it is often necessary to add high percentages of salt(s) to these oral care compositions to achieve the desired qualities and/or level of technical effectiveness. The use of salts at the levels required to achieve the desired qualities and/or level of technical effectiveness imparts a perceived level of saltiness within the mouth of the end user during or following the use of the product, which is often undesirable and unpleasant for the end user. For example, the perception of a salty taste during tooth brushing or of a salty aftertaste after tooth brushing is completed, can cause certain consumers either to not try a particular oral care product or to try such oral care product for a limited duration and discontinue use. This is especially an issue for “baking soda” type toothpastes, which can contain percentages of sodium bicarbonate as high as 75% by weight.

There have been numerous attempts to formulate consumer products comprising high salt levels, such as oral care products, and particularly baking soda type toothpastes and toothpowders, to address this issue. In certain prior art compositions, high percentages of artificial sweetener are added to product. This can be undesirable to the end user, such when the individual desires to reduce his or her overall intake of artificial sweeteners. Other prior art oral care compositions included bittering agents, solubilizing agents, and oil-like materials such as castor oil, triglycerides, and seed or flower oils to combat the ordinarily imparted salty taste and/or aftertaste of such consumer products.

Although the use of cooling materials has been previously described generally in, e.g., U.S. Pat. Nos. 6,890,567; 8,071,531; 10,494,330; and 8,377,458, each of which is incorporated herein by reference, there has been no study which cooling materials can be effective for reducing saltiness in a consumer product.

Accordingly, there remains a need and demand for orally administered, imbibed or ingested consumer products, particularly oral care compositions that ordinarily impart a salty taste and/or after taste to the mouth of the end user wherein the salty taste and/or aftertaste is reduced or inhibited.

### SUMMARY OF THE INVENTION

Thus, the present disclosure is directed to oral care compositions and/or other orally administered, ingested or imbibed consumer products comprising salts and certain cooling materials or combinations thereof, in which the salty taste ordinarily imparted by the salts perceived during or after use of the consumer product has been reduced or inhibited.

In one aspect, the disclosure features an orally administered, imbibed, or ingested consumer product including: (i) at least about 5% by weight of one or more salts; and (ii) at least one cooling material selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof; wherein the at least one cooling material is present in an amount effective to reduce or inhibit the salty taste or after-taste ordinarily imparted by the salts to the mouth of an end user.

In some embodiments, the one or more salts is selected from the group consisting of metallic carbonate salts; metallic chloride salts; metallic fluoride salts; metallic phosphate salts; stannous salts; strontium salts; zinc salts; and combinations thereof. In some embodiments, the one or more salts is selected from the group consisting of disodium phosphate; disodium ethylenediaminetetraacetic acid; monosodium glutamate; potassium bicarbonate; potassium chloride; sodium bicarbonate; sodium acetate; sodium alginate; sodium ascorbate; sodium benzoate; sodium caseinate; sodium chloride; sodium citrate; sodium dehydroacetate; sodium diacetate; sodium erythorbate; sodium hydroxide; sodium lactate; sodium nitrate; sodium nitrite; sodium polyphosphate; sodium propionate; sodium saccharin; sodium stearoyl lactylate; sodium sulfite; tetrasodium pyrophosphate; trisodium phosphate; stannous fluoride; strontium chloride; zinc acetate; zinc chloride; zinc citrate; zinc fluoride; zinc gluconate; zinc lactate; zinc salicylate; zinc sulphate; and combinations thereof.

In certain embodiments, the one or more salts is sodium bicarbonate. In further embodiments, the one or more salts is sodium chloride.

In some embodiments, the consumer product includes at least 10% by weight of the one or more salts; at least about 20% by weight of the one or more salts; at least about 40% by weight of the one or more salts; at least about 50% by weight of the one or more salts; or at least about 60% by weight of the one or more salts.

In particular embodiments, the consumer product is an oral care composition. Oral care compositions as disclosed herein include, by way of non-limiting example, toothpastes and tooth powders.

In another aspect, the present disclosure features an orally administered, imbibed, or ingested consumer product including: (i) at least 5% by weight of at least one salt selected from the group consisting of metallic carbonate salts; metallic chloride salts; metallic fluoride salts; metallic phosphate salts; stannous salts; strontium salts; zinc salts; and combinations thereof, wherein said at least one salt ordinarily imparts a salty taste to the mouth of the end user of said consumer product; and (ii) at least one cooling material selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof, wherein said at least one cooling material is present in an amount effective to reduce or inhibit the salty taste of the consumer product ordinarily imparted by the at least one salts.

In some embodiments, the at least one cooling material is present in an amount between about 0.0001% to about 1%, between about 0.0005% and about 1%, between about 0.001% and about 0.3%, between about 0.01% and 2% by weight, between about 0.01% and 1.5% by weight, between about 0.01 and 1% by weight, between about 0.01% and 0.5% by weight, or between about 0.05% and 0.2% by weight of the consumer product.

In some embodiments, the consumer product is an oral care composition. In certain embodiments, the consumer product is a toothpaste or a rinse. In further embodiments, the consumer product is a tooth powder.

In another aspect, the present disclosure features a cooling material for use in reducing or inhibiting salty taste or after-taste imparted by one or more salts to the mouth of an end user in an orally administered, imbibed, or ingested consumer product, wherein the consumer product includes: (i) at least about 5% by weight of the one or more salts; and (ii) the cooling material, which is selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof.

In some embodiments, the one or more salts is selected from the group consisting of metallic carbonate salts; metallic chloride salts; metallic fluoride salts; metallic phosphate salts; stannous salts; strontium salts; zinc salts; and combinations thereof.

In some embodiments, the one or more salts is selected from the group consisting of disodium phosphate; disodium ethylenediaminetetraacetic acid; monosodium glutamate; potassium bicarbonate; potassium chloride; sodium bicarbonate; sodium acetate; sodium alginate; sodium ascorbate; sodium benzoate; sodium caseinate; sodium chloride; sodium citrate; sodium dehydroacetate; sodium diacetate; sodium erythorbate; sodium hydroxide; sodium lactate; sodium nitrate; sodium nitrite; sodium polyphosphate; sodium propionate; sodium saccharin; sodium stearoyl lactylate; sodium sulfite; tetrasodium pyrophosphate; trisodium phosphate; stannous fluoride; strontium chloride; zinc acetate; zinc chloride; zinc citrate; zinc fluoride; zinc gluconate; zinc lactate; zinc salicylate; zinc sulphate; and combinations thereof.

In particular embodiments, the one or more salts is sodium bicarbonate. In other embodiments, the one or more salts is sodium chloride.

In some embodiments, the consumer product includes at least 10% by weight of the one or more salts; at least about 20% by weight of the one or more salts; at least about 40% by weight of the one or more salts; at least about 50% by weight of the one or more salts; or at least about 60% by weight of the one or more salts.

In certain embodiments, the consumer product is an oral care composition.

In another aspect, the present disclosure features a cooling material for use in reducing or inhibiting the salty taste of an orally administered, imbibed, or ingested consumer product ordinarily imparted by at least one salt, wherein the consumer product includes: (i) at least 5% by weight of the at least one salt, which is selected from the group consisting of metallic carbonate salts; metallic chloride salts; metallic fluoride salts; metallic phosphate salts; stannous salts; strontium salts; zinc salts; and combinations thereof and (ii) the cooling material, which is selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof.

In some embodiments, the at least one cooling material is present in an amount between about 0.0001% to about 1%, between about 0.0005% and about 1%, between about 0.001% and about 0.3%, between about 0.01% and 2% by weight, between about 0.01% and 1.5% by weight, between about 0.01 and 1% by weight, between about 0.01% and 0.5% by weight, or between about 0.05% and 0.2% by weight of the consumer product.

In certain embodiments, the consumer product is an oral care composition. In some embodiments, the consumer product is a toothpaste or a rinse. In other embodiments, the consumer product is a tooth powder.

In another aspect, the present disclosure features a use of a cooling material in an orally administered, imbibed, or ingested consumer product for reducing or inhibiting salty taste or after-taste imparted by one or more salts to the mouth of an end user, wherein the consumer product includes: (i) at least about 5% by weight of the one or more salts; and (ii) the cooling material, which is selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof.

In some embodiments, the one or more salts is selected from the group consisting of metallic carbonate salts; metallic chloride salts; metallic fluoride salts; metallic phosphate salts; stannous salts; strontium salts; zinc salts; and combinations thereof.

In certain embodiments, the one or more salts is selected from the group consisting of disodium phosphate; disodium ethylenediaminetetraacetic acid; monosodium glutamate; potassium bicarbonate; potassium chloride; sodium bicarbonate; sodium acetate; sodium alginate; sodium ascorbate; sodium benzoate; sodium caseinate; sodium chloride; sodium citrate; sodium dehydroacetate; sodium diacetate; sodium erythorbate; sodium hydroxide; sodium lactate; sodium nitrate; sodium nitrite; sodium polyphosphate; sodium propionate; sodium saccharin; sodium stearoyl lactylate; sodium sulfite; tetrasodium pyrophosphate; trisodium phosphate; stannous fluoride; strontium chloride; zinc acetate; zinc chloride; zinc citrate; zinc fluoride; zinc gluconate; zinc lactate; zinc salicylate; zinc sulphate; and combinations thereof.

In certain embodiments, the one or more salts is sodium bicarbonate. In other embodiments, the one or more salts is sodium chloride.

In some embodiments, the consumer product includes at least 10% by weight of the one or more salts; at least about 20% by weight of the one or more salts; at least about 40% by weight of the one or more salts; at least about 50% by weight of the one or more salts; or at least about 60% by weight of the one or more salts.

In some embodiments, the consumer product is an oral care composition. The oral composition can be, for example, a toothpaste, a rinse, or a tooth powder.

In another aspect, the present disclosure features a use of a cooling material in an orally administered, imbibed, or ingested consumer product for reducing or inhibiting the salty taste of the consumer product ordinarily imparted by at least one salt, wherein the consumer product includes: (i) at least 5% by weight the at least one salt, which is selected from the group consisting of metallic carbonate salts; metallic chloride salts; metallic fluoride salts; metallic phosphate salts; stannous salts; strontium salts; zinc salts; and combinations thereof; and (ii) the cooling material, which is selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof.

In some embodiments, the cooling material is present in an amount between about 0.0001% to about 1%, between about 0.0005% and about 1%, between about 0.001% and about 0.3%, between about 0.01% and 2% by weight, between about 0.01% and 1.5% by weight, between about 0.01 and 1% by weight, between about 0.01% and 0.5% by weight, or between about 0.05% and 0.2% by weight of the consumer product. In some embodiments, the consumer product is an oral care composition. In further embodiments, the consumer product is a toothpaste, a rinse, or a tooth powder.

In a further aspect, the present disclosure features a method of reducing or inhibiting the salty taste and/or aftertaste of an orally administered, imbibed, or ingested consumer product including at least about 5% by weight of one or more salt, the method including: adding to the consumer product a cooling material selected from one of at least one cooling material selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof in an amount effective to reduce or inhibit the salty taste or after-taste ordinarily imparted by the one or more salt.

In some embodiments, the one or more salt ordinarily imparts a salty taste to the mouth of the end user of the consumer product.

In some embodiments, the one or more salt is selected from the group consisting of metallic carbonate salts; metallic chloride salts; metallic fluoride salts; metallic phosphate salts; stannous salts; strontium salts; zinc salts; and combinations thereof.

In particular embodiments, the one or more salt is sodium bicarbonate. In other embodiments, the one or more salt is sodium chloride.

In some embodiments, the one or more salt is present in an amount of at least 10% by weight; at least about 20% by weight; at least about 40% by weight; at least about 50% by weight; or at least about 60% by weight of the consumer product.

In some embodiments, the at least one cooling material is added in an amount between about 0.0001% to about 1%, between about 0.0005% and about 1%, between about 0.001% and about 0.3%, between about 0.01% and 2% by weight, between about 0.01% and 1.5% by weight, between about 0.01 and 1% by weight, between about 0.01% and 0.5% by weight, or between about 0.05% and 0.2% by weight of the consumer product.

In further embodiments, the consumer product is an oral care composition, e.g., a toothpaste, a rinse, or a tooth powder.

These and other objects of the present disclosure will become apparent from the following detailed description.

### DETAILED DESCRIPTION

The present disclosure features, inter alia, consumer products that are orally administered, ingested, or imbibed that include one or more salts and one or more cooling materials, as well as related compositions, methods, and uses. The cooling material is present in the consumer product in an amount effective to reduce or inhibit the salty taste of the consumer product, or the salty taste or after-taste ordinary imparted by the salts to the mouth of the end user.

### Definitions

The terms used in this specification generally have their ordinary meanings in the art, within the context of this disclosure and in the specific context where each term is used. Certain terms are discussed below, or elsewhere in the specification, to provide additional guidance to a person of ordinary skill in the art describing the compounds, compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” a plurality, and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “flavor composition,” “flavor,” or “flavoring” refers to a composition that contains one or more flavor compounds (e.g., co-ingredients) that provide a desired taste when combined with a solvent and that is suitable for oral administration and oral consumption. Additionally, flavoring can also refer to an overall flavor composition that comprises two or more “sub” flavor compositions. Non-limiting examples of flavor co-ingredients that are generally included in flavor compositions are listed in S. Arctander, Perfume and Flavor Chemicals, 1969, Montclair, N.J., USA or any of its more recent versions, each of which are incorporated herein by reference. The skilled person in the art of flavor creation is able to select flavor co-ingredients on the basis of general knowledge and according to the nature of the end product to be flavored and the desired taste.

As used herein, the term “oral care composition” refers to a type of consumer product used by an end user to clean, refresh, preserve, or provide treatment to the teeth and/or gums of the individual (or animal). Oral care compositions are not ordinarily ingested, imbibed or otherwise consumed by the end user, but are used exclusively in the mouth cavity and then spit out or rinsed out.

As used herein, the phrase “consumer product” refers to composition that is in a form ready for use by the end user for the marketed indication. A solvent suitable for use in a consumer product is a solvent that, when combined with other components of the consumer product, will not render the consumer product unfit for its intended end use. As used herein, the phrase “consumer product” includes, without limitation, foods, beverages, oral care compositions, prescription and over-the-counter pharmaceutical products, nutraceuticals, and dietary supplements. “End users” can be humans or animals, and in the case of animals, “consumer products” can, by way of non-limiting example, include animal feed or feed supplements.

As used herein, the term “effective quantity” or “effective amount” will be defined as the amount of the presently disclosed cooling materials or combinations thereof in a consumer product, or in a flavor composition added to a consumer product, in which the cooling materials or combinations thereof will contribute to the reduction or inhibition of the salty taste ordinarily associated with the consumer product.

### Salts and Salt Content

The salt(s) comprised in the consumer products of the present disclosure can be any salt known by one skilled in the art to provide desired qualities or technical effectiveness to an oral care composition or other consumer product (e.g., abrasiveness for tooth cleaning or polishing, the reduction in the growth of pathogens and organisms and prevent spoilage of food or beverages, or to improve the texture of the consumer product, such as improving the tenderness and palatability of leaner cuts of meat). Non-limiting examples of such salts include metallic carbonate salts (e.g., potassium bicarbonate, sodium bicarbonate); metallic chloride salts (e.g., potassium chloride, sodium chloride); metallic fluoride salts (e.g., sodium fluoride, stannous fluoride); metallic phosphate salts (e.g., sodium polyphosphate, tetrasodium pyrophosphate); stannous salts (e.g., stannous fluoride); strontium salts (e.g., strontium chloride); zinc salts (e.g., zinc acetate, zinc chloride, zinc citrate, zinc fluoride, zinc gluconate, zinc lactate, zinc salicylate, zinc sulphate); and any combination(s) thereof. In certain embodiments, the salt is selected from the group consisting of disodium phosphate; disodium ethylenediaminetetraacetic acid; monosodium glutamate; potassium bicarbonate; potassium chloride; sodium bicarbonate; sodium acetate; sodium alginate; sodium ascorbate; sodium benzoate; sodium caseinate; sodium chloride; sodium citrate; sodium dehydroacetate; sodium diacetate; sodium erythorbate; sodium hydroxide; sodium lactate; sodium nitrate; sodium nitrite; sodium polyphosphate; sodium propionate; sodium saccharin; sodium stearoyl lactylate; sodium sulfite; tetrasodium pyrophosphate; trisodium phosphate; stannous fluoride; strontium chloride; zinc acetate; zinc chloride; zinc citrate; zinc fluoride; zinc gluconate; zinc lactate; zinc salicylate; zinc sulphate; and any combinations thereof. In particular embodiments, the salt is sodium bicarbonate or sodium chloride.

The weight percentage of salt(s) contained in an oral care composition or other consumer product, which ordinarily imparts a salty taste and/or after taste in the mouth of the end user, can vary depending upon the type of consumer product and the selection of salt(s) comprised in the consumer product formulation. For example, in certain non-limiting examples, consumer products, e.g., oral care compositions, ordinarily imparting a salty taste and/or aftertaste to the mouth and typically considered to have a high percentage of salts can comprise between about 0.5% and about 75% by weight of one or more salt(s), or between about 5% and about 75% by weight of one or more salt(s), or between about 10% and about 70% by weight one or more salts, or between about 10% and about 60% by weight of one or more salts, or between about 20% and about 50% by weight of one or more salts, or between about 30% to about 40% by weight of one or more salts. In further examples, the consumer product, e.g., oral care composition, can comprise between about 1% and about 60% by weight, or between about 1% and about 50% by weight, or between about 1% and 40% by weight, or between about 1% and about 30% by weight, or between about 1% and about 15% by weight, or between about 1% and about 10% by weight, or between about 2% and about 15% by weight, or between about 2% and about 30% by weight, or between about 5% and about 60% by weight of one or more salts, or between about 5% and about 50% by weight of one or more salts, or between about 5% and about 40% by weight of one or more salts, or between about 5% and about 30% by weight of one or more salts, or between about 5% and about 25% by weight of one or more salts, or between about 5% and about 15% by weight of one or more salts, or between about 10% and 75% by weight of one or more salts, or between about 10% and about 50% by weight of one or more salts, or between about 10% and about 40% by weight of one or more salts, or between about 10% and about 30% by weight of one or more salts, or between about 10% and about 25% by weight of one or more salts. In additional examples, the consumer product, e.g., oral care composition, can comprise between about 30% to about 75% by weight of one or more salts, or between about 30% and about 60% by weight of one or more salts, or between about 30% and about 50% by weight of one or more salts, or between about 20% and about 75% by weight of one or more salts, or between about 20% and about 60% by weight of one or more salts, or between about 20% and about 40% by weight of one or more salts, or between about 20% and about 30% by weight of one or more salts. In yet further examples, the consumer product, e.g., oral care composition, can comprise between about 40% and about 75% by weight of one or more salts, or between about 45% and about 75% by weight of one or more salts, or between about 40% and about 60% by weight of one or more salts, or between about 50% and about 75% by weight of one or more salts, or between about 50% and about 70% by weight of one or more salts, or between about 50% and about 60% by weight of one or more salts.

For instance, in some embodiments, the consumer product, e.g., oral care composition, comprises sodium bicarbonate. The sodium bicarbonate can be present in the consumer product, e.g., oral care composition, in an amount between about 1% and about 75% by weight of one or more salt(s), or between about 5% and 75% by weight, between about 40% and 60% by weight, or between about 50% and 60% by weight, or between about 10% and about 70% by weight one or more salts, or between about 10% and about 60% by weight, or between about 20% and about 50% by weight of one or more salts, or between about 30% to about 40% by weight. In further examples, the sodium bicarbonate can be present in the consumer product, e.g., oral care composition, in an amount between about 1% and about 60% by weight, or between about 1% and about 50% by weight, or between about 1% and 40% by weight, or between about 1% and about 30% by weight, or between about 1% and about 15% by weight, or between about 1% and about 10% by weight, or between about 2% and about 15% by weight, or between about 2% and about 30% by weight, or between about 5% and about 60% by weight, or between about 5% and about 50% by weight, or between about 5% and about 40% by weight, or between about 5% and about 30% by weight, or between about 5% and about 25% by weight, or between about 5% and about 15% by weight, or between about 10% and 75% by weight, or between about 10% and about 50% by weight, or between about 10% and about 40% by weight, or between about 10% and about 30% by weight, or between about 10% and about 25% by weight. In additional examples, the sodium bicarbonate can be present in the consumer product, e.g., oral care composition, in an amount between about 30% to about 75% by weight, or between about 30% and about 60% by weight, or between about 30% and about 50% by weight, or between about 20% and about 75% by weight, or between about 20% and about 60% by weight, or between about 20% and about 40% by weight, or between about 20% and about 30% by weight. In certain embodiments, the oral care product can be a toothpaste that can comprise up to 75% of sodium bicarbonate. In certain embodiments, the oral care product can be a tooth powder that can comprise up to 95% of sodium bicarbonate. In yet further examples, the sodium bicarbonate can be present in the consumer product, e.g., oral care composition, in an amount between about 40% and about 75% by weight, or between about 45% and about 70% by weight, or between about 40% and about 60% by weight, or between about 50% and about 75% by weight, or between about 50% and about 70% by weight, or between about 50% and about 60% by weight of one or more salts. In particular embodiments, the sodium bicarbonate is present in a toothpaste or toothpowder.

In further embodiments, the oral care composition or other consumer product comprises sodium chloride. The sodium chloride can be present in the oral care composition or other consumer product in an amount between 0.5% and about 75% by weight of one or more salt(s), or between about 5% and 75% by weight, between about 40% and 60% by weight, or between about 50% and 60% by weight, or between about 10% and about 70% by weight one or more salts, or between about 10% and about 60% by weight, or between about 20% and about 50% by weight of one or more salts, or between about 30% to about 40% by weight. In further examples, the sodium chloride is present in an oral care composition in an amount between about 0.5% and about 60% by weight, or between about 0.5% and about 50% by weight, or between about 0.5% and 40% by weight, or between about 0.5% and about 30% by weight, or between about 0.5% and about 15% by weight, or between about 0.5% and about 10% by weight, or between about 1% and about 10% by weight, or between about 1% and about 15% by weight, or between about 2% and about 30% by weight, or between about 5% and about 60% by weight, or between about 5% and about 50% by weight, or between about 5% and about 40% by weight, or between about 5% and about 30% by weight, or between about 5% and about 25% by weight, or between about 5% and about 15% by weight, or between about 10% and 75% by weight, or between about 10% and about 50% by weight, or between about 10% and about 40% by weight, or between about 10% and about 30% by weight, or between about 10% and about 25% by weight of one or more salts. In particular embodiments, the sodium chloride is present in a toothpaste or toothpowder in an amount between about 0.5% and about 20% by weight, or between about 0.5% and 5% by weight; or between about 1% and about 7.5% by weight; or between about 1% and about 15% by weight, or between about 1% and about 10% by weight.

In addition to the above disclosure, one of ordinary skill in the art can recognize the total amount of salt in a consumer product that can impart a salty taste and/or aftertaste to the consumer product when consumed or ingested by the end user.

### Cooling Materials and Combinations Thereof

It was surprisingly discovered that consumer products ordinarily imparting a salty taste to the mouth can have a reduced or inhibited imparted salty taste and/or aftertaste when including certain cooling materials compared to the consumer products without the certain cooling materials described herein. The consumer product can be, for example, an oral care composition. Said cooling materials can be incorporated into the consumer products (e.g., oral care compositions), or into a flavor composition that is added to a consumer product. The cooling materials effective in reducing or inhibiting the salty taste and/or aftertaste are referred to herein as “cooling materials capable of reducing or inhibiting saltiness.” Cooling materials outside of those disclosed herein capable of reducing saltiness are referred to as “additional cooling materials.”

For illustration, and not as a limitation, the term “reduced” as used herein with respect to imparted salty taste and/or aftertaste refers to one or both of the following:

(i) the oral care composition or other consumer product comprising a cooling material capable of reducing or inhibiting saltiness or a cooling combination when used or consumed by the end user is perceived by the end user as having an imparted salty taste and/or aftertaste that is less salty when compared to a similarly formulated oral care composition or consumer product that does not comprise the presently disclosed cooling materials or cooling combinations; and/or

(ii) the oral care composition or other consumer product comprising a cooling material capable of reducing or inhibiting saltiness or a cooling combinations when used or consumed by the end user is perceived by the end user as imparting a salty taste and/or aftertaste substantially equivalent to a similarly formulated oral care composition or other consumer product comprising a lower weight percentage of salt(s).

By way of an illustrative but non-limiting example, a toothpaste or tooth powder comprising 40% by weight of sodium bicarbonate according to the present disclosure can be perceived by an end user as imparting a salty taste and/or aftertaste substantially equivalent to a similarly formulated toothpaste or tooth powder comprising 25% by weight of sodium bicarbonate that is not formulated according to the present disclosure.

The cooling materials capable of reducing or inhibiting saltiness of the present disclosure include, but are not limited to, p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof. The cooling material capable of reducing or inhibiting saltiness can be present in the consumer product in an effective amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2%, or between about 0.05% and about 0.3% by weight of the consumer product. For example, in certain embodiments, the effective amount of the cooling material capable of reducing or inhibiting saltiness is between about 0.0008% and about 0.39% by weight of the consumer product.

In further embodiments of the present disclosure, the cooling material capable of reducing or inhibiting saltiness is selected from the group consisting of N-(3-Hydroxy-4-methoxyphenyl)-2-isopropyl-5,5-dimethylcyclohexanecarboxamide, N-(4-(Cyanomethyl)phenyl)-2-isopropyl-5,5-dimethylcyclohexanecarboxamide, (1R,2S,5R)—N-(4-(cyanomethyl)phenyl)menthylcarboxamide, and (1R,2S,5R)—N-(2-(pyridine-2-yl)ethyl)menthylcarboxamide, and combinations thereof. These cooling materials can be present in a consumer product in combination with any of the other cooling materials disclosed herein. In certain embodiments, these cooling materials can be present in the consumer product in an effective amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2%, or between about 0.05% and about 0.3% by weight of the consumer product.

The present disclosure also features cooling combinations that can be incorporated into a consumer product. As used herein, a “cooling combination” refers to a combination of two or more of the presently disclosed cooling material capable of reducing or inhibiting saltiness, or a combination of at least one cooling material capable of reducing or inhibiting saltiness and at least one additional cooling material.

In certain embodiments, one or more of the presently disclosed cooling materials capable of reducing or inhibiting saltiness (i.e., p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof) can be combined with at least one additional cooling material selected from the group consisting of 1-isopulegol; N-ethyl-5-methyl-2-propan-2-ylcyclohexane-1-carboxamide; N-[(ethoxycarbonyl)methyl)-p-menthane-3-carboxamide; 2-isopropyl-N,2,3-trimethylbutyramide; menthyl lactate; menthol; natural and/or artificial peppermint oil(s); natural and/or artificial spearmint oil(s); and combinations thereof. Non-limiting examples of natural or artificial peppermint oils for use as additional cooling materials of the present invention include peppermint oil Willamette; peppermint oil Midwest; peppermint oil Idaho; peppermint oil Yakima; peppermint oil Madras; peppermint oil Terpeneless; Mentha Arvensis; Mentha Arvensis Terpeneless; Mintact® N&A peppermint type, and combinations thereof.

In some embodiments, a cooling combination of the present disclosure comprises 3-L-menthoxypropan-1,2-diol and 1-(2-hydroxy-4-methylcyclohexyl)-ethanone. In certain embodiments, the cooling composition comprises between about 1% to about 50% by weight of the cooling combination of 3-L-menthoxypropan-1,2-diol and between about 1% to about 50% by weight of 1-(2-hydroxy-4-methylcyclohexyl)-ethanone. In another such embodiment, the cooling combination comprises between about 25% to about 75% by weight of menthol, and between about 1% to about 25% by weight of 1-(2-hydroxy-4-methylcyclohexyl)-ethanone.

In other embodiments, a cooling combination of the present disclosure comprises one or more natural and/or artificial peppermint oil(s), menthol, and 1-(2-hydroxy-4-methylcyclohexyl)-ethanone. In certain embodiments, the cooling combination comprises between about 20% to about 60% by weight of the cooling combination of one or more natural and/or artificial peppermint oil(s); between about 25% to about 60% by weight of menthol, and between about 1% to about 25% by weight of 1-(2-hydroxy-4-methylcyclohexyl)-ethanone.

The cooling material(s) and/or cooling compositions of the present disclosure can be added to a flavor composition that is subsequently added to the oral care composition or other consumer product to reduce and/or inhibit the ordinarily imparted salty taste of the consumer product; or the presently disclosed cooling material(s) and/or cooling compositions can be added directly into the product base formulation of the oral care composition or other consumer product to reduce or inhibit the ordinarily imparted salty taste of the consumer product without otherwise substantially changing the existing flavor profile of such oral care composition or other consumer product.

### Consumer Products Having a Reduced or Inhibited Salty Taste Profile

Certain consumer products, e.g., oral care compositions, can ordinarily impart a salty taste and/or after taste to the mouth of the end user due to the salt content comprised therein. Non-limiting examples of such consumer products, e.g., oral care compositions, include toothpaste, toothpowders, mouthwash, mouth rinses, breath mints, breath sprays, and film strips for halitosis. Other consumer products, e.g., oral care compositions, that can impart a salty taste and/or after taste are known to those of ordinary skill in the art.

Furthermore, in addition to oral care compositions, certain other orally administered, imbibed, or ingested consumer products can also impart a salty taste and/or after taste to the mouth of the end user due to the salt content comprised therein. The imparted salty taste and/or after taste of the consumer product can cause the end user not to try such consumer product or to discontinue or limit the use of such product. This can be particularly problematic if such consumer product is a pharmaceutical product required by the end user to maintain or achieve certain health benefits. Accordingly, the present disclosure provides including one or more cooling materials capable of reducing or inhibiting saltiness in the consumer products disclosed above, where the cooling material in the consumer product reduces the salty taste and/or aftertaste to the mouth of the end user that is ordinarily imparted by the salts present in the consumer product.

In addition to the consumer products described above, the present disclosure also includes consumer products such as foods or beverages, animal feed and pet foods, pharmaceuticals (including over-the-counter medications), nutraceuticals, and dietary supplements. Non-limiting examples of suitable food and/or beverage products ordinarily imparting a salty taste and/or after taste to the mouth include, e.g., canned vegetables or vegetable juices; canned beans; canned or dehydrated soup, broth or bouillon, including seasoned ramen noodle mixes; frozen dinner entrees; frozen pizzas or breaded meats; prepackaged mixes for potatoes, rice, pasta, or stuffing; prepared pasta or tomato sauces; cured, salted or canned meat, fish or poultry (i.e., deli meats, sardines, sausage, hot dogs) and prepared marinades, condiments, seasonings, dips and salad dressings, including mixes for the preparation thereof. Non-limiting examples of suitable pharmaceuticals include, e.g., headache and heartburn medications.

For example, in some embodiments, the cooling material or cooling combination of the present disclosure is added directly into the consumer product base formulation, thereby allowing for the reduction or inhibition of the ordinarily imparted salty taste of the consumer product without otherwise substantially changing the existing flavor profile of such oral care composition or other consumer product.

Flavor Compositions Comprising Cooling Materials

In other embodiments, the presently disclosed cooling material capable of reducing or inhibiting saltiness or cooling combination is added to the consumer product formulation as a part of a flavor composition that is subsequently added into the consumer product formulation. Therefore, the cooling material or combinations thereof embodied in the presently disclosed subject matter can be employed in more complex flavor composition comprising one or more other flavor co-ingredients, or other flavor compositions, to modify the overall taste characteristics of the flavor composition via their own organoleptic properties or through enhancing or complimenting the contributions of the other flavor ingredient(s) present within the said composition, including other compounds known to reduce or inhibit saltiness. Flavor compositions according to the disclosed subject matter can be in the form of a simple mixture of the presently disclosed cooling materials or cooling compositions, various flavor co-ingredients, adjuvants, and solvents, or in the form of a biphasic system such as an emulsion or microemulsion. Such systems are well-known to persons skilled in the art.

Flavor compositions of the present disclosure comprise from about 0.01% to about 100% by weight, from about 0.05% to about 50% by weight, from about 0.1% to about 30% by weight, or from about 0.5% to about 30% by weight of the presently disclosed cooling materials or cooling combinations based on the total weight of the flavor composition into which the presently disclosed cooling materials or cooling compositions are incorporated. For example, in certain non-limiting embodiments, the flavor composition can comprise from about 0.05% to about 70%, from about 0.05% to about 50%, from about 0.1% to about 30%, from about 0.1% to about 20%, from about 0.1% to about 10%, from about 0.1% to about 5%, from about 0.1% to about 1%, from about 0.5% to about 30%, from about 0.5% to about 20%, from about 0.5% to about 10%, from about 0.5% to about 5%, from about 0.5% to about 1%, from about 1% to about 30%, from about 1% to about 20%, from about 1% to about 10%, from about 1% to about 5%, from about 5% to about 30%, from about 5% to about 20%, from about 5% to about 15%, from about 5% to about 10%, from about 10% to about 50%, from about 10% to about 40%, from about 10% to about 30%, from about 10% to about 20%, from about 20% to about 30%, from about 20% to about 30%, from about 20% to about 40%, from about 30% to about 50%, from about 40% to about 50%, or from about 40% to about 70%, from about 50% to about 70%, from about 40% to about 65%, from about 50% to about 65%, from about 55% to about 65% by weight of the one or more of the presently disclosed cooling materials capable of reducing saltiness or cooling combination

In some embodiments, one or more of the cooling material capable of reducing or inhibiting saltiness selected from p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide can be present in a flavor composition that is added to a consumer product. The cooling material(s) capable of reducing or inhibiting saltiness is can be present in the flavor composition in an amount of from about 0.05% to about 70% by weight, from about 0.05% to about 50% by weight, from about 0.1% to about 30% by weight, or from about 0.5% to about 30% by weight of the presently disclosed cooling materials or cooling combinations based on the total weight of the flavor composition into which the presently disclosed cooling materials capable of reducing or inhibiting saltiness are incorporated. For example, in certain non-limiting embodiments, the flavor composition can comprise from about 0.05% to about 70% by weight, from about 0.05% to about 50%, from about 0.1% to about 30%, from about 0.1% to about 20%, from about 0.1% to about 10%, from about 0.1% to about 5%, from about 0.1% to about 1%, from about 0.5% to about 30%, from about 0.5% to about 20%, from about 0.5% to about 10%, from about 0.5% to about 5%, from about 0.5% to about 1%, from about 1% to about 30%, from about 1% to about 20%, from about 1% to about 10%, from about 1% to about 5%, from about 5% to about 30%, from about 5% to about 20%, from about 5% to about 15%, from about 5% to about 10%, from about 10% to about 50%, from about 10% to about 40%, from about 10% to about 30%, from about 10% to about 20%, from about 20% to about 30%, from about 20% to about 30%, from about 20% to about 40%, from about 30% to about 50%, or from about 40% to about 50%, from about 40% to about 70%, from about 50% to about 70%, from about 40% to about 65%, from about 50% to about 65%, from about 55% to about 65% of the presently disclosed cooling materials capable of reducing or inhibiting saltiness by weight of the flavor composition.

In certain embodiments, the presently disclosed cooling material capable of reducing or inhibiting saltiness is p-menthane-3,8-diol. In certain embodiments, the presently disclosed cooling material capable of reducing or inhibiting saltiness is hydroxyethyl-menthyl ether. In certain embodiments, the presently disclosed cooling material capable of reducing or inhibiting saltiness is 3-L-menthoxypropan-1-2-diol. In certain embodiments, the presently disclosed cooling material capable of reducing or inhibiting saltiness is L-menthyl-3-hydroxybutanoate. In certain embodiments, the presently disclosed cooling material capable of reducing or inhibiting saltiness is 1-(2-hydroxy-4-methylcyclohexyl)-ethanone. In certain embodiments, the presently disclosed cooling material capable of reducing or inhibiting saltiness is N-(4-methoxyphenyl)-p-menthanecarboxamide.

In some embodiments, the flavor composition can comprise from about 0.05% to about 50%, from about 0.1% to about 30%, from about 0.1% to about 20%, from about 0.1% to about 10%, from about 0.1% to about 5%, from about 0.1% to about 1%, from about 0.5% to about 30%, from about 0.5% to about 20%, from about 0.5% to about 10%, from about 0.5% to about 5%, from about 0.5% to about 1%, from about 1% to about 30%, from about 1% to about 20%, from about 1% to about 10%, from about 1% to about 5%, from about 5% to about 30%, from about 5% to about 20%, from about 5% to about 15%, from about 5% to about 10% of the one or more of the presently disclosed cooling materials capable of reducing saltiness or cooling combination selected from the group consisting of p-menthane-3,8-diol; hydroxyethyl-menthyl ether; 3-L-menthoxypropan-1-2-diol; L-menthyl-3-hydroxybutanoate; N-(4-methoxyphenyl)-p-menthanecarboxamide; and combinations thereof. In some embodiments, the flavor composition can additionally comprise from about 1% to about 25%, from about 1% to about 20%, from about 1% to about 15%, from about 1% to about 10%, from about 5% to about 25%, from about 5% to about 20%, from about 5% to about 15%, from about 10% to about 25%, or from about 10% to about 15% by weight of 1-(2-hydroxy-4-methylcyclohexyl)-ethanone.

In some embodiments, a flavor composition of the present disclosure can comprise one or more cooling materials capable of reducing or inhibiting saltiness selected from the group consisting of N-(3-Hydroxy-4-methoxyphenyl)-2-isopropyl-5,5-dimethylcyclohexanecarboxamide, N-(4-(Cyanomethyl)phenyl)-2-isopropyl-5,5-dimethylcyclohexanecarboxamide, (1R,2S,5R)—N-(4-(cyanomethyl)phenyl)menthylcarboxamide, and (1R,2S,5R)—N-(2-(pyridine-2-yl)ethyl)menthylcarboxamide, and combinations thereof. These cooling materials can be present in a flavor composition in combination with any of the other cooling materials disclosed herein. In certain embodiments, these cooling materials can be present in the flavor composition in an amount of from about 0.05% to about 70% by weight, from about 0.05% to about 50% by weight, from about 0.1% to about 30% by weight, or from about 0.5% to about 30% by weight of the presently disclosed cooling materials or cooling combinations based on the total weight of the flavor composition into which the presently disclosed cooling materials capable of reducing or inhibiting saltiness are incorporated. For example, in certain non-limiting embodiments, the flavor composition can comprise from about 0.05% to about 70% by weight, from about 0.05% to about 50%, from about 0.1% to about 30%, from about 0.1% to about 20%, from about 0.1% to about 10%, from about 0.1% to about 5%, from about 0.1% to about 1%, from about 0.5% to about 30%, from about 0.5% to about 20%, from about 0.5% to about 10%, from about 0.5% to about 5%, from about 0.5% to about 1%, from about 1% to about 30%, from about 1% to about 20%, from about 1% to about 10%, from about 1% to about 5%, from about 5% to about 30%, from about 5% to about 20%, from about 5% to about 15%, from about 5% to about 10%, from about 10% to about 50%, from about 10% to about 40%, from about 10% to about 30%, from about 10% to about 20%, from about 20% to about 30%, from about 20% to about 30%, from about 20% to about 40%, from about 30% to about 50%, or from about 40% to about 50%, from about 40% to about 70%, from about 50% to about 70%, from about 40% to about 65%, from about 50% to about 65%, from about 55% to about 65% of the presently disclosed cooling materials capable of reducing or inhibiting saltiness by weight of the flavor composition.

In other embodiments, a flavor composition of the present disclosure, in addition to one or more of p-menthane-3,8-diol, hydroxyethyl-menthyl ether, 3-L-menthoxypropan-1-2-diol; L-menthyl-3-hydroxybutanoate, 1-(2-hydroxy-4-methylcyclohexyl)-ethanone, and/or N-(4-methoxyphenyl)-p-menthanecarboxamide, comprises 1-isopulegol, N-ethyl-5-methyl-2-propan-2-ylcyclohexane-1-carboxamide, N-[(ethoxycarbonyl)methyl)-p-menthane-3-carboxamide, 2-isopropyl-N,2,3-trimethylbutyramide, menthyl lactate, menthol, natural and/or artificial peppermint oil(s), natural and/or artificial spearmint oil(s), and combinations thereof. In certain embodiments, the flavor composition comprises from about 0.05% to about 70%, from about 0.05% to about 50%, from about 0.1% to about 30%, from about 0.1% to about 20%, from about 0.1% to about 10%, from about 0.1% to about 5%, from about 0.1% to about 1%, from about 0.5% to about 30%, from about 0.5% to about 20%, from about 0.5% to about 10%, from about 0.5% to about 5%, from about 0.5% to about 1%, from about 1% to about 30%, from about 1% to about 20%, from about 1% to about 10%, from about 1% to about 5%, from about 5% to about 30%, from about 5% to about 20%, from about 5% to about 15%, from about 5% to about 10%, from about 10% to about 50%, from about 10% to about 40%, from about 10% to about 30%, from about 10% to about 20%, from about 20% to about 30%, from about 20% to about 30%, from about 20% to about 40%, from about 30% to about 50%, from about 40% to about 50%, or from about 40% to about 70%, from about 50% to about 70%, from about 40% to about 65%, from about 50% to about 65%, from about 55% to about 65% by weight of 1-isopulegol, N-ethyl-5-methyl-2-propan-2-ylcyclohexane-1-carboxamide, N-[(ethoxycarbonyl)methyl)-p-menthane-3-carboxamide, 2-isopropyl-N,2,3-trimethylbutyramide, menthyl lactate, menthol, natural and/or artificial peppermint oil(s), natural and/or artificial spearmint oil(s), and combinations thereof in addition to one or more of p-menthane-3,8-diol, hydroxyethyl-menthyl ether, 3-L-menthoxypropan-1-2-diol, L-menthyl-3-hydroxybutanoate, 1-(2-hydroxy-4-methylcyclohexyl)-ethanone and N-(4-methoxyphenyl)-p-menthanecarboxamide.

In certain embodiments, the cooling combination comprises between about 20% to about 60% by weight of the cooling combination of one or more natural and/or artificial peppermint oil(s); between about 25% to about 60% by weight of menthol, and between about 1% to about 25% by weight of 1-(2-hydroxy-4-methylcyclohexyl)-ethanone.

In some embodiments, the flavor composition comprises between about 0.1% to about 30% by weight of 1-(2-hydroxy-4-methylcyclohexyl)-ethanone). In other embodiments, the flavor composition comprises 1-(2-hydroxy-4-methylcyclohexyl)-ethanone) and a second cooling material of the present disclosure selected from the group consisting of p-menthane-3,8-diol; hydroxyethyl-menthyl ether; 3-L-menthoxypropan-1-2-diol; L-menthyl-3-hydroxybutanoate; N-(4-methoxyphenyl)-p-menthanecarboxamide, and combinations thereof. In yet further embodiments, the flavor composition comprises between about 5% to about 15% by weight of 1-(2-hydroxy-4-methylcyclohexyl)-ethanone) and between about 0% to about 5% by weight of a second cooling material of the present disclosure selected from the group consisting of p-menthane-3,8-diol; hydroxyethyl-menthyl ether; 3-L-menthoxypropan-1-2-diol; L-menthyl-3-hydroxybutanoate; N-(4-methoxyphenyl)-p-menthanecarboxamide, and combinations thereof.

In further embodiments, the flavor composition comprises 1-(2-hydroxy-4-methylcyclohexyl)-ethanone) and at least one additional cooling material selected from the group consisting of 1-isopulegol; N-ethyl-5-methyl-2-propan-2-ylcyclohexane-1-carboxamide; N-[(ethoxycarbonyl)methyl)-p-menthane-3-carboxamide; 2-isopropyl-N,2,3-trimethylbutyramide; menthyl lactate; menthol; natural and/or artificial peppermint oil(s); natural and/or artificial spearmint oils; and combinations thereof. In yet further embodiments, the flavor composition comprises between about 5% to about 15% by weight of 1-(2-hydroxy-4-methylcyclohexyl)-ethanone) and between about 50% to about 75% by weight of at least one additional cooling material selected from the group consisting of 1-isopulegol; N-ethyl-5-methyl-2-propan-2-ylcyclohexane-1-carboxamide; N-[(ethoxycarbonyl)methyl)-p-menthane-3-carboxamide; 2-isopropyl-N,2,3-trimethylbutyramide; menthyl lactate; menthol; natural and/or artificial peppermint oil(s); natural and/or artificial spearmint oils; and combinations thereof.

The flavor compositions of the presently disclosed subject matter can further comprise one or more support materials. By way of non-limiting example, support materials can include diluents, e.g., ethanol, purified water, glycerol; solvents; carriers, e.g., propylene glycol, triacetin; preservatives, e.g., sulfites, sodium nitrite, propionic acid, sorbic acid, benzoic acid, disodium ethylenediaminetetraacetic acid (EDTA); flavoring agents, e.g., alcohols, esters, aldehydes; ketones, lactones, phenols, flavor enhancers, e.g., monosodium glutamate (MSG), monopotassium glutamate, calcium diglutamate (CDG), guanosine monophosphate disodium guanylate, sodium guanylate, inosinic acid and its salts, L-leucine; antioxidants, e.g., ascorbic acid, sodium ascorbate, fatty acid esters of ascorbic acid, tocopherols, alpha-tocopherol, gamma-tocopherol, delta-tocopherol, propyl gallate, octyl gallate, erythorbic acid, sodium erythorbate, dodecyl gallate, tertiary-butyl hydroquinone (TBHQ), butylated hydroxyanisole (BHA), butylated hydroxytoluene (BHT), citric acid, 4-hexylresorcinol; color retention agents; dyes or lakes; sequestrants, emulsifiers, e.g., lecithins, monoglyceride, acetylated monoglyceride, lactylated monoglyceride, sugar ester, sorbitan ester, polyglycerol ester, calcium stearoyl di laciate; stabilizers; acids, bases, and/or anticaking agents, e.g., calcium silicate, magnesium carbonate, sodium aluminosilicate. Nonlimiting examples of solvents commonly used in flavors are also known in the art and include, but are not limited to: water, medium-chain triglycerides (MCTs), propylene glycol, triacetin, triethyl citrate, benzyl alcohol, benzyl benzoate, ethanol, vegetable oils, and terpenes. The flavor compositions according to the disclosed subject matter can be in an encapsulated form, e.g., a flavor composition entrapped into a solid matrix that can comprise wall-forming and plasticizing materials such as mono-, di-, or trisaccharides, natural or modified starches, hydrocolloids, cellulose derivatives, polyvinyl acetates, polyvinylalcohols, proteins, or pectins. Examples of particularly useful matrix materials include, for example, sucrose, glucose, lactose, levulose, fructose, maltose, ribose, dextrose, isomalt, sorbitol, mannitol, xylitol, lactitol, maltitol, pentatol, arabinose, pentose, xylose, galactose, maltodextrin, dextrin, chemically modified starch, hydrogenated starch hydrolysate, succinylated or hydrolysed starch, agar, carrageenan, gum arabic, gum accacia, tragacanth, alginates, methyl cellulose, carboxymethyl cellulose, hydroxyethyl cellulose, hydroxypropylmethyl cellulose, derivatives, gelatin, agar, alginate, and mixtures thereof. Encapsulation is well-known to persons skilled in the art, and can be performed, for instance, using techniques such as spray-drying, agglomeration or extrusion, or coating encapsulation, including coacervation and complex coacervation techniques.

Flavor composition can be added in an amount of from between about 0.5% to about 5.0% by weight of the consumer product as described herein. Flavor composition can be added in an amount of from between about 0.5% to about 3.0%. For example, the consumer product can comprise about 0.5%, about 0.8%, about 0.9%, about 1%, about 1.1%, about 1.2%, about 1.3%, about 1.4%, about 1.5%, about 1.8%, about 2.0%, about 2.5%, or about 3.0% by weight of the flavor composition disclosed herein. In another example, the consumer product can comprise between about 0.8% to about 2.0% by weight of the flavor composition, between about 0.9% and about 1.8% by weight of the flavor composition, between about 1% and about 1.6% by weight of the flavor composition, or between about 1.1% or about 1.5% by weight of the flavor composition. In certain non-limiting embodiments, the consumer product is an oral care composition. The oral care composition can be, for instance, a toothpaste.

By way of a non-limiting example for oral care compositions, an effective amount of a flavor comprising the presently disclosed cooling materials or combinations thereof for a mouth rinse ranges from about 0.02% to about 5%; from about 0.02% to about 3%; from about 0.02% to about 1%; from about 0.02% to about 0.7%; or from about 0.02% to about 0.5%; from about 0.02% to about 0.25% by weight of the mouth rinse; and an effective amount of a flavor comprising the presently disclosed cooling materials or cooling combinations for a toothpaste or tooth powder ranges from about 0.2% to about 2%; or from about 0.5% to about 1.5% by weight of the toothpaste or tooth powder.

Cooling Materials in Consumer Products

Those skilled in the art can employ the desired level of said cooling materials or cooling combinations to provide the desired flavor and impart the reduced and/or inhibited salt taste and/or aftertaste of the consumer product. However, typical concentrations of the presently disclosed cooling materials or cooling compositions effective to reduce or inhibit the perceived salty taste and/or aftertaste of a consumer product range from about 0.01% to about 2% by weight based on the total weight of the consumer product composition into which the compound is incorporated.

By way of a non-limiting example for oral care compositions, an effective amount of the presently disclosed cooling materials or combinations thereof a toothpaste or tooth powder ranges from about 0.0001% to about 2.0%, or 0.001% to about 2.0%, or about 0.01% to about 2.0% by weight or from about 0.05% to about 1.5% by weight of the toothpaste.

For example, described herein is an orally administered, imbibed, or ingested consumer product ordinarily imparting a salty taste and/or after taste to the mouth of the end user, e.g., an oral care composition, which comprises:

(i) at least about 5% by weight of one or more salts; at least 10% by weight of one or more salts; at least about 20% by weight of one or more salts; at least about 40% by weight of one or more salts; at least about 50% by weight of one or more salts; at least about 60% by weight of one or more salts; or at least about 70% by weight of one or more salts; and

(ii) at least one cooling material selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof, wherein the cooling material is present in an amount effective to reduce or inhibit the salty taste and/or after-taste which would ordinarily be imparted by the salt(s) to the mouth of the end user.

In some embodiments, the consumer product, e.g., oral care composition, comprises at least about 5% by weight of sodium bicarbonate. In some embodiments, the consumer product, e.g., oral care composition, comprises at least 10% by weight of sodium bicarbonate. In some embodiments, the consumer product, e.g., oral care composition, comprises at least about 20% by weight of sodium bicarbonate. In some embodiments, the consumer product, e.g., oral care composition, comprises at least about 40% by weight of sodium bicarbonate. In some embodiments, the consumer product, e.g., oral care composition, comprises at least about 50% by weight of sodium bicarbonate. In further embodiments, the consumer product, e.g., oral care composition, comprises at least about 60% by weight of sodium bicarbonate. In other embodiments, the consumer product, e.g., oral care composition, comprises at least about 70% by weight of sodium bicarbonate.

In other embodiments, the consumer product, e.g., oral care composition, comprises at least about 5% by weight of sodium chloride. In some embodiments, the consumer product, e.g., oral care composition, comprises at least 10% by weight of sodium chloride. In some embodiments, the consumer product, e.g., oral care composition, comprises at least about 20% by weight of sodium chloride. In some embodiments, the consumer product, e.g., oral care composition, comprises at least about 40% by weight of sodium chloride. In some embodiments, the consumer product, e.g., oral care composition, comprises at least about 50% by weight of sodium chloride. In further embodiments, the consumer product, e.g., oral care composition, comprises at least about 60% by weight of sodium chloride. In other embodiments, the consumer product, e.g., oral care composition, comprises at least about 70% by weight of sodium chloride.

In another example, described herein is a consumer product, e.g., an oral care composition, having a salty taste and/or aftertaste, wherein the consumer product comprises:

at least 5% by weight of one or more salts selected from the group consisting of metallic carbonate salts; metallic chloride salts; metallic fluoride salts; metallic phosphate salts; stannous salts; strontium salts; zinc salts; and combinations thereof, wherein said salt ordinarily imparts a salty taste to the mouth of the end user of said consumer product; and

at least one cooling material selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof,

wherein said at least one cooling material is comprised in an amount effective to reduce or inhibit the salty taste of the consumer product ordinarily imparted by the salt(s), wherein such effective amount is between about 0.0001% to about 5%, from about 0.0005% to about 5%, from about 0.0001% to about 4%, from about 0.0005% to about 4%, from about 0.0001 to about 3%, from about 0.0005 to about 3%, from about 0.0001% to about 2%, from about 0.0005% to about 2%, from about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2%, or between about 0.05% and about 0.3% by weight of the consumer product. For example, in certain embodiments, the effective amount of the at least one cooling material is between about 0.0008% and about 0.39% by weight of the consumer product.

In some embodiments, the consumer product, e.g., oral care composition, comprises p-menthane-3,8-diol in an amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2% by weight of the consumer product. In some embodiments, the consumer product, e.g., oral care composition, comprises hydroxyethyl-menthyl ether in an amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2% by weight of the consumer product. In some embodiments, the consumer product, e.g., oral care composition, comprises 3-L-menthoxypropan-1-2-diol in an amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2% by weight of the consumer product. In some embodiments, the consumer product, e.g., oral care composition, comprises L-menthyl-3-hydroxybutanoate in an amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2% by weight of the consumer product. In some embodiments, the consumer product, e.g., oral care composition, comprises 1-(2-hydroxy-4-methylcyclohexyl)-ethanone in an amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2% by weight of the consumer product. In some embodiments, the consumer product, e.g., oral care composition, comprises N-(4-methoxyphenyl)-p-menthanecarboxamide in an amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2% by weight of the consumer product.

In yet further embodiments, the consumer product, e.g., oral care composition, comprises a combination of two or more of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; and N-(4-methoxyphenyl)-p-menthanecarboxamide in an amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2% by weight of the consumer product.

In further embodiments, the consumer product, comprises at least one cooling material selected from the group consisting of N-(3-Hydroxy-4-methoxyphenyl)-2-isopropyl-5,5-dimethylcyclohexanecarboxamide, N-(4-(Cyanomethyl)phenyl)-2-isopropyl-5,5-dimethylcyclohexanecarboxamide, (1R,2S,5R)—N-(4-(cyanomethyl)phenyl)menthylcarboxamide, and (1R,2S,5R)—N-(2-(pyridine-2-yl)ethyl)menthylcarboxamide, and combinations thereof. In certain embodiments, N-(3-Hydroxy-4-methoxyphenyl)-2-isopropyl-5,5-dimethylcyclohexanecarboxamide can be present in the consumer product in an effective amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2%, or between about 0.05% and about 0.3% by weight of the consumer product. In certain embodiments N-(4-(Cyanomethyl)phenyl)-2-isopropyl-5,5-dimethylcyclohexanecarboxamide can be present in the consumer product in an effective amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2%, or between about 0.05% and about 0.3% by weight of the consumer product. In certain embodiments, (1R,2S,5R)—N-(4-(cyanomethyl)phenyl)menthylcarboxamide can be present in the consumer product in an effective amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2%, or between about 0.05% and about 0.3% by weight of the consumer product. In certain embodiments, (1R,2S,5R)—N-(2-(pyridine-2-yl)ethyl)menthylcarboxamide can be present in the consumer product in an effective amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2%, or between about 0.05% and about 0.3% by weight of the consumer product. In certain embodiments, two or more of N-(3-Hydroxy-4-methoxyphenyl)-2-isopropyl-5,5-dimethylcyclohexanecarboxamide, N-(4-(Cyanomethyl)phenyl)-2-isopropyl-5,5-dimethylcyclohexanecarboxamide, (1R,2S,5R)—N-(4-(cyanomethyl)phenyl)menthylcarboxamide, and (1R,2S,5R)—N-(2-(pyridine-2-yl)ethyl)menthylcarboxamide can be present in the consumer product in an effective amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2%, or between about 0.05% and about 0.3% by weight of the consumer product.

In further embodiments, the consumer product, e.g., oral care composition, comprises at least about 5% by weight of sodium bicarbonate. In some embodiments, the consumer product, e.g., oral care composition, comprises at least 10% by weight of sodium bicarbonate. In some embodiments, the consumer product, e.g., oral care composition, comprises at least about 20% by weight of sodium bicarbonate. In some embodiments, the consumer product, e.g., oral care composition, comprises at least about 40% by weight of sodium bicarbonate. In some embodiments, the consumer product, e.g., oral care composition, comprises at least about 50% by weight of sodium bicarbonate. In further embodiments, the consumer product, e.g., oral care composition, comprises at least about 60% by weight of sodium bicarbonate. In other embodiments, the consumer product, e.g., oral care composition, comprises at least about 70% by weight of sodium bicarbonate.

In other embodiments, the consumer product, e.g., oral care composition, comprises at least about 5% by weight of sodium chloride. In some embodiments, the consumer product, e.g., oral care composition, comprises at least 10% by weight of sodium chloride. In some embodiments, the consumer product, e.g., oral care composition, comprises at least about 20% by weight of sodium chloride. In some embodiments, the consumer product, e.g., oral care composition, comprises at least about 40% by weight of sodium chloride. In some embodiments, the consumer product, e.g., oral care composition, comprises at least about 50% by weight of sodium chloride. In further embodiments, the consumer product, e.g., oral care composition, comprises at least about 60% by weight of sodium chloride. In other embodiments, the consumer product, e.g., oral care composition, comprises at least about 70% by weight of sodium chloride.

In particular embodiments, the oral care composition is a toothpaste comprising a salt selected from the group consisting of sodium bicarbonate, sodium chloride, and combinations thereof. In some embodiments, the toothpaste comprises between about 5% and 70% by weight, between about 10% and 60% by weight, or between about 20% and 50% by weight of sodium bicarbonate; and between about 0.05% and 1% by weight, or between about 0.05% and 0.2% by weight of one or more cooling materials selected from the group consisting of p-menthane-3,8-diol; hydroxyethyl-menthyl ether, 3-L-menthoxypropan-1-2-diol, L-menthyl-3-hydroxybutanoate, 1-(2-hydroxy-4-methylcyclohexyl)-ethanone), N-(4-methoxyphenyl)-p-menthanecarboxamide, and combinations thereof. In other embodiments, the toothpaste comprises between about 5% and 70% by weight, between about 10% and 60% by weight, or between about 20% and 50% by weight of sodium bicarbonate and between about 0.01% and 1% by weight, or between about 0.01% and 0.5% by weight of 1-(2-hydroxy-4-methylcyclohexyl)-ethanone). In further embodiments, the oral composition is a toothpowder comprising a salt selected from the group consisting of sodium bicarbonate, sodium chloride, and combinations thereof. In some embodiments, the toothpowder comprises between about 5% and 70% by weight, between about 10% and 60% by weight, or between about 20% and 50% by weight of sodium bicarbonate; and between about 0.05% and 1% by weight, or between about 0.05% and 0.2% by weight of one or more cooling materials selected from the group consisting of p-menthane-3,8-diol; hydroxyethyl-menthyl ether, 3-L-menthoxypropan-1-2-diol, L-menthyl-3-hydroxybutanoate, 1-(2-hydroxy-4-methylcyclohexyl)-ethanone), N-(4-methoxyphenyl)-p-menthanecarboxamide, and combinations thereof. In other embodiments, the toothpowder comprises between about 5% and 70% by weight, between about 10% and 60% by weight, or between about 20% and 50% by weight of sodium bicarbonate and between about 0.01% and 1% by weight, or between about 0.01% and 0.5% by weight of 1-(2-hydroxy-4-methylcyclohexyl)-ethanone).

In certain embodiments, the oral care composition is a toothpaste or a toothpowder. The toothpaste or toothpowder can comprise other typical toothpaste base materials known to those skilled in the art. Non-limiting examples of toothpaste base materials include abrasives (e.g., calcium carbonate, calcium pryrophosphate, magnesium carbonate, dicalcium phosphate, dicalcium phosphate dihydrate, hydrated aluminum hydroxide, silica, silica hydrate,); humectant (e.g., sorbitol, glycerol, propylene glycol, polyethylene glycol, glycerin); water; detergents (e.g.,: sodium lauryl sulfate, sodium lauryl sarcosinate, sodium alkylsulfo succinate, sodium cocomonoglyceride sulfonate, sucrose fatty acid esters), rheology modifiers (e.g., sodium carboxymethylcellulose, sodium alginate, carrageenan, xanthan gum, cellulose gum, sodium polyacrylate, bentonite, laponite, carbomers); active ingredients (e.g., anti-caries agents such as sodium fluoride, sodium monofluorophosphate, stannous fluoride; anti-plaque agents such as triclosan; anti-dentine hypersensitivity agents such as potassium salts, whitening agents such as papain, calcium peroxide, hydrogen peroxide, tetra sodium pyrophosphate, and titanium dioxide); sweeteners (e.g., stevia, sucralose, saccharine, sodium saccharin, xylitol); colorants; and preservatives (e.g., sodium benzoate, ethyl paraben, methyl paraben). The toothpaste can also contain other flavor materials (e.g., cinnamon, eucalyptus; 1-isopulegol; N-ethyl-5-methyl-2-propan-2-ylcyclohexane-1-carboxamide; N-[(ethoxycarbonyl)methyl)-p-menthane-3-carboxamide; 2-isopropyl-N,2,3-trimethylbutyramide; menthyl lactate; menthol; natural and/or artificial peppermint oil(s); natural and/or artificial spearmint oils).

Vranic et al., “Formulation Ingredients for Toothpastes and Mouthwashes,” Bosnian Journal of Basic Medical Sciences. 4(4):51-58, 2004; and Moharamzadeh et al., “Biocompatibility of oral care products,” Biocompatibility of Dental Biomaterials, 2017 both describe toothpaste formulations, and the contents of each of the references are incorporated herein by reference in their entireties. By way of non-limiting example, a toothpaste formulation can include from about 20% to about 40% by weight water; up to about 50% by weight abrasives; fluoride (e.g., in an amount of about 1450 ppm); from about 0.5% to about 2.0% by weight of detergents, such as sodium lauryl sulfate. Additionally, a toothpaste formulation of the present disclosure can include antibacterial agents, flavorants, remineralizers, humectants (e.g., glycerol, xylitol, sorbitol, polyethylene glycol, polypropylene glycol); antisensitivity agents (e.g., strontium chloride, potassium nitrate, arginine); and/or anticalculus agents (e.g., sodium polyphosphate; zinc citrate). One of ordinary skill in the art would be able to recognize a suitable toothpaste formulation that can include one of the cooling materials capable of reducing or inhibiting saltiness as disclosed herein.

### Method of Reducing or Inhibiting the Salty Taste of an Oral Care Composition

Furthermore, described herein is a method of reducing or inhibiting the salty taste of a consumer product, e.g., an oral care composition, comprising:

adding one or more of the cooling materials capable of reducing or inhibiting saltiness to the consumer product, e.g., oral care composition, in an amount effective to reduce or inhibit the salty taste (e.g., as compared to a similarly formulated oral care composition or other consumer product), wherein the consumer product, e.g., oral care composition, comprises at least about 20% by weight, at least about 30% by weight, at least about 40% by weight, at least about 50% by weight, or at least about 60% by weight of one or more salts selected from the group consisting of metallic carbonate salts, metallic chloride salts, metallic fluorinde salts, metallic phosphate salts, stannous salts, strontium salts, zinc salts, and combinations thereof. The cooling material added to the consumer product, e.g., oral care composition, is selected from p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof. The cooling material can be added to the consumer product, e.g., oral care composition, in an amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2% by weight of the consumer product.

For example, in some embodiments, the methods disclosed herein include reducing or inhibiting the salty taste of a toothpaste, the method comprising: adding one or more of the cooling materials capable of reducing or inhibiting saltiness to the toothpaste in an amount effective to reduce or inhibit the salty taste (e.g., as compared to a similarly formulated oral care composition or other consumer product). The toothpaste can comprise at least about 20% by weight, at least about 30% by weight, at least about 40% by weight, at least about 50% by weight, or at least about 60% by weight of one or more salts selected from the group consisting of metallic carbonate salts, metallic chloride salts, metallic fluorinde salts, metallic phosphate salts, stannous salts, strontium salts, zinc salts, and combinations thereof. The cooling material that is added to the toothpaste is selected from p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof. The cooling material can be added to the toothpaste in an amount between about 0.0001% to about 1%, between about 0.0005% and about 1%, between about 0.0005% and about 0.005%, between about 0.0005% and about 0.05%, between about 0.0005% and about 0.5%, between about 0.001% and about 0.4%, between about 0.001% and about 0.3%, between about 0.001% and about 0.2%, between about 0.001% and about 0.1%, between about 0.005% and about 0.1%, between about 0.01% and about 0.1%, between about 0.01% and about 2% by weight, between about 0.01% and about 1.5% by weight, between about 0.01 and about 1% by weight, between about 0.01% and about 0.5% by weight, or between about 0.05% and about 0.2% by weight of the toothpaste.

According to the methods disclosed herein, the consumer product, e.g., oral care composition, can furthermore include at least one additional cooling material selected from the group consisting of 1-isopulegol; N-ethyl-5-methyl-2-propan-2-ylcyclohexane-1-carboxamide; N-[(ethoxycarbonyl)methyl)-p-menthane-3-carboxamide; 2-isopropyl-N,2,3-trimethylbutyramide; menthyl lactate; menthol; natural and/or artificial peppermint oil(s); and combinations thereof. The natural or artificial peppermint oils for use as additional cooling materials of the present invention include peppermint oil Willamette; peppermint oil Midwest; peppermint oil Idaho; peppermint oil Yakima; peppermint oil Madras; peppermint oil Terpeneless; Mentha Arvensis; Mentha Arvensis Terpeneless; Mintact® N&A peppermint type, and combinations thereof. In particular non-limiting embodiments, the consumer product is a toothpaste.

Furthermore, according to the methods disclosed herein, the cooling material can be added to the consumer product as a “cooling combination,” which refers to a combination of two or more of the presently disclosed cooling materials, or a combination of at least one of the present disclosed cooling materials and at least one additional cooling material. In particular non-limiting embodiments, the consumer product is a toothpaste.

In some embodiments, a cooling combination of the present disclosure comprises 3-L-menthoxypropan-1,2-diol and 1-(2-hydroxy-4-methylcyclohexyl)-ethanone. In certain embodiments, the cooling composition comprises between about 1% to about 50% by weight of the cooling combination of 3-L-menthoxypropan-1,2-diol and between about 1% to about 50% by weight of 1-(2-hydroxy-4-methylcyclohexyl)-ethanone. In further embodiments, a cooling combination of the present disclosure comprises between about 25% to about 75% by weight of menthol and between about 1% to about 25% by weight of 1-(2-hydroxy-4-methylcyclohexyl)-ethanone.

In other embodiments, a cooling combination of the present disclosure comprises one or more natural and/or artificial peppermint oil(s), menthol, and 1-(2-hydroxy-4-methylcyclohexyl)-ethanone. In certain embodiments, the cooling combination comprises between about 20% to about 60% by weight of the cooling combination of one or more natural and/or artificial peppermint oil(s); between about 25% to about 60% by weight of menthol, and between about 1% to about 25% by weight of 1-(2-hydroxy-4-methylcyclohexyl)-ethanone.

In some embodiments of the methods disclosed herein, the consumer product, e.g., oral care composition comprises sodium bicarbonate. In particular non-limiting embodiments, the consumer product is a toothpaste. The sodium bicarbonate can be present in the oral care composition in an amount of between about 1% and about 75% by weight of one or more salt(s), or between about 5% and 75% by weight, between about 40% and 60% by weight, or between about 50% and 60% by weight, or between about 10% and about 70% by weight one or more salts, or between about 10% and about 60% by weight, or between about 20% and about 50% by weight of one or more salts, or between about 30% to about 40% by weight. In further examples, the sodium bicarbonate can be present in the consumer product, e.g., oral care composition, in an amount between about 1% and about 60% by weight, or between about 1% and about 50% by weight, or between about 1% and 40% by weight, or between about 1% and about 30% by weight, or between about 1% and about 15% by weight, or between about 1% and about 10% by weight, or between about 2% and about 15% by weight, or between about 2% and about 30% by weight, or between about 5% and about 60% by weight, or between about 5% and about 50% by weight, or between about 5% and about 40% by weight, or between about 5% and about 30% by weight, or between about 5% and about 25% by weight, or between about 5% and about 15% by weight, or between about 10% and 75% by weight, or between about 10% and about 50% by weight, or between about 10% and about 40% by weight, or between about 10% and about 30% by weight, or between about 10% and about 25% by weight of one or more salts. In additional examples, the sodium bicarbonate can be present in the consumer product, e.g., oral care composition, in an amount between about 30% to about 75% by weight, or between about 30% and about 60% by weight, or between about 30% and about 50% by weight, or between about 20% and about 75% by weight, or between about 20% and about 60% by weight, or between about 20% and about 40% by weight, or between about 20% and about 30% by weight of one or more salts. In yet further examples, the sodium bicarbonate can be present in the consumer product, e.g., oral care composition, in an amount between about 40% and about 75% by weight, or between about 45% and about 70% by weight, or between about 40% and about 60% by weight, or between about 50% and about 75% by weight, or between about 50% and about 70% by weight, or between about 50% and about 60% by weight of one or more salts.

In further embodiments of the methods disclosed herein, the consumer product, e.g., oral care composition, comprises sodium chloride. The sodium chloride can be present in the oral care composition in an amount between about 1% and about 75% by weight of one or more salt(s), or between about 5% and 75% by weight, between about 40% and 60% by weight, or between about 50% and 60% by weight, or between about 10% and about 70% by weight one or more salts, or between about 10% and about 60% by weight, or between about 20% and about 50% by weight of one or more salts, or between about 30% to about 40% by weight. In further examples, the sodium chloride can be present in the consumer product, e.g., oral care composition, in an amount between about 1% and about 60% by weight, or between about 1% and about 50% by weight, or between about 1% and 40% by weight, or between about 1% and about 30% by weight, or between about 1% and about 15% by weight, or between about 1% and about 10% by weight, or between about 2% and about 15% by weight, or between about 2% and about 30% by weight, or between about 5% and about 60% by weight, or between about 5% and about 50% by weight, or between about 5% and about 40% by weight, or between about 5% and about 30% by weight, or between about 5% and about 25% by weight, or between about 5% and about 15% by weight, or between about 10% and 75% by weight, or between about 10% and about 50% by weight, or between about 10% and about 40% by weight, or between about 10% and about 30% by weight, or between about 10% and about 25% by weight of one or more salts. In additional examples, the sodium chloride can be present in the consumer product, e.g., oral care composition, in an amount between about 30% to about 75% by weight, or between about 30% and about 60% by weight, or between about 30% and about 50% by weight, or between about 20% and about 75% by weight, or between about 20% and about 60% by weight, or between about 20% and about 40% by weight, or between about 20% and about 30% by weight of one or more salts. In yet further examples, the sodium chloride can be present in the consumer product, e.g., oral care composition, in an amount between about 40% and about 75% by weight, or between about 45% and about 75% by weight, or between about 40% and about 60% by weight, or between about 50% and about 75% by weight, or between about 50% and about 70% by weight, or between about 50% and about 60% by weight of one or more salts.

### EXAMPLES

The present application is further described by means of the examples, presented below, wherein the abbreviations have the usual meaning in the art.

The use of such examples is illustrative only and does not limit the scope and meaning of the disclosed subject matter or of any exemplified term. Likewise, the disclosed subject matter is not limited to any particularly preferred embodiments described herein. Indeed, many modifications and variations of the disclosed subject matter are apparent to those skilled in the art upon reading this specification. The disclosed subject matter is therefore to be limited only by the terms of the appended claims along with the full scope of equivalents to which the claims are entitled.

The following Examples are provided as specific embodiments of the present invention, wherein the abbreviations have the usual meaning in the art.

### Example 1: Comparison of Cooling Materials in a Toothpaste

A sample of a commercially available toothpaste containing approximately 50% by weight of sodium bicarbonate was used as a Control. In addition, toothpaste samples containing the certain cooling materials were also prepared by adding 0.02% of each cooling material to the commercially available sodium bicarbonate toothpaste. A listing of the samples is provided in Table 1-1. All of the toothpaste samples, including the Control, were evaluated by an expert panel comprised of 4 Flavorist(s), 2 Flavor Lab Managers, and 1 Flavor Lab Technician. Each sample was assigned a number by each evaluator to indicate saltiness, using a saltiness scale of 1(less salty) to 7(most salty). The average saltiness rating for each of the toothpaste samples was calculated and is shown in Table 1-1.

As shown in the Table, the toothpaste samples comprising the cooling materials of the present invention (i.e., p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone); N-(4-methoxyphenyl)-p-menthanecarboxamide) were perceived as having a less salty taste when compared with both the Control and the other tested cooling materials.

TABLE 1-1

| | | Average |
|---|---|---|
| TOOTHPASTE SAMPLE | % | RATING |
| Control | N/A | 6.43 |
| N-ethyl-5-methyl-2-propan-2-ylcyclohexane-1- | 0.2 | 6.57 |
| carboxamide | | |
| Menthyl Lactate | 0.2 | 6.43 |
| N-[(ethoxycarbonyl)methyl)-p-menthane-3- | 0.2 | 6.43 |
| carboxamide | | |
| 1-isopulegol | 0.2 | 6.29 |
| 2-isopropyl-N,2,3-trimethylbutyramide | 0.2 | 6 |
| p-menthane-3,8-diol | 0.2 | 5.86 |
| hydroxyethyl-menthyl ether | 0.2 | 5.57 |
| N-(4-methoxyphenyl)-p-menthanecarboxamide | 0.2 | 5.57 |
| L-menthyl-3-hydroxybutanoate | 0.2 | 3.86 |
| 3-L-menthoxypropan-1,2-diol | 0.2 | 3.00 |
| 1-(2-hydroxy-4-methylcyclohexyl)-ethanone | 0.2 | 2.43 |

### Example 2

### Example 2A: Exemplary Flavor Composition

An exemplary flavor composition of the present disclosure is shown in Table 2-1. As shown in the Table, the flavor composition comprises about 90% by weight of a cooling combination of the present disclosure, the cooling combination comprising menthol, peppermint oil(s), and 1-(2-hydroxy-4-methylcyclohexyl-ethanone).

TABLE 2-1

| Flavor Composition | WEIGHT % |
|---|---|
| menthol | 40 |
| peppermint oils | 40 |
| 1-(2-hydroxy-4-methylcyclohexyl)-ethanone | 10 |
| anethole | 10 |
| TOTAL | 100% |

### Example 2B: Exemplary Toothpaste Composition

A exemplary baking soda type toothpaste of the present disclosure comprising the flavor composition of Example 2A is shown in Table 2-2.

TABLE 2-2

| Toothpaste Composition | WEIGHT % |
|---|---|
| Exemplary Flavor Composition of Example 2A | 0.5-2.0% |
| Toothpaste Base (20% sodium bicarbonate) | 98-99.5% |
| TOTAL | 100% |

### Example 2C: Exemplary Toothpaste Composition

A exemplary baking soda type toothpaste of the present disclosure comprising 1-(2-hydroxy-4-methylcyclohexyl)-ethanone is shown in Table 2-3.

TABLE 2-3

| Toothpaste Composition | WEIGHT % |
|---|---|
| 1-(2-hydroxy-4-methylcyclohexyl)-ethanone | 0.01-0.3% |
| Sodium Bicarbonate | 0.1-65% |
| Humectants | 20-35% |
| Water | 15-25% |
| Detergents | 1-2% |
| Rheology Modifiers | 3% |
| Colorants | 1-2% |
| Flavor | 1-2% |
| Active Ingredient(s) | 0.1-0.15% |
| Sweeteners | 0.01-1.5% |

### Example 3

### Example 3A: Flavor Composition

An flavor composition of the present disclosure was made as shown in Table 3-1.

TABLE 3-1

| Flavor Composition | WEIGHT % |
|---|---|
| 1-(2-hydroxy-4-methylcyclohexyl)-ethanone | 7 |
| Other cooling materials capable of reducing or | 54 |
| inhibiting saltiness in addition to 1-(2-hydroxy-4- | |
| methylcyclohexyl)-ethanone | |
| Other flavor materials | 39 |
| TOTAL | 100% |

### Example 3B: Toothpaste Composition

A baking soda type toothpaste of the present disclosure comprising the flavor composition of Example 3A was made as shown in Table 3-2.

TABLE 3-2

| Toothpaste Composition | WEIGHT % |
|---|---|
| Flavor Composition of Example 3A | 1.4 |
| Toothpaste base (35% by weight sodium | 98.6 |
| bicarbonate) | |
| TOTAL | 100% |

A study with 35 panelists was conducted to assess the efficacy of the cooling material for reducing or inhibiting saltiness in a toothpaste. The panelists were instructed to brush their teeth for 60 seconds with the toothpaste composition described in Table 3-2. Of the 35 panelists, 27 panelists found the toothpaste composition to be just about right, only somewhat salty, or not salty at all, while only 8 panelists found the toothpaste composition to be too salty or somewhat too salty.

### Example 4

### Example 4A: Flavor Composition

An flavor composition of the present disclosure was made as shown in Table 4-1.

TABLE 4-1

| Flavor Composition | WEIGHT % |
|---|---|
| 1-(2-hydroxy-4-methylcyclohexyl)-ethanone | 5% |
| Other cooling materials capable of reducing or | 4.8% |
| inhibiting saltiness in addition to 1-(2-hydroxy-4- | |
| methylcyclohexyl)-ethanone | |
| Additional cooling materials | 63.1% |
| Other flavor materials | 27.1% |
| TOTAL | 100% |

### Example 4B: Toothpaste Composition

A baking soda type toothpaste of the present disclosure comprising the flavor composition of Example 4A was made as shown in Table 4-2.

TABLE 4-2

| Toothpaste Composition | WEIGHT % |
|---|---|
| Flavor Composition of Example 4A | 1.4 |
| Toothpaste base (35% by weight sodium | 98.6 |
| bicarbonate) | |
| TOTAL | 100% |

A study with 35 panelists was conducted to assess the efficacy of the cooling material for reducing or inhibiting saltiness in a toothpaste. The panelists were instructed to brush their teeth for 60 seconds with the toothpaste composition described in Table 4-2. Of the 35 panelists, 26 panelists found the toothpaste composition to be just about right, only somewhat salty, or not salty at all, while only 9 panelists found the toothpaste composition to be too salty or somewhat too salty.

### Example 5

### Example 5A: Flavor Composition

An flavor composition of the present disclosure was made as shown in Table 5-1.

TABLE 5-1

| Flavor Composition | WEIGHT % |
|---|---|
| 1-(2-hydroxy-4-methylcyclohexyl)-ethanone | 5% |
| Other cooling materials capable of reducing or | 2.85% |
| inhibiting saltiness | |
| Additional cooling materials | 66.149% |
| Other flavor materials | 26% |
| TOTAL | 100% |

### Example 5B: Toothpaste Composition

A baking soda type toothpaste of the present disclosure comprising the flavor composition of Example 5A was made as shown in Table 5-2.

TABLE 5-2

| Toothpaste Composition | WEIGHT % |
|---|---|
| Flavor Composition of Example 5A | 1.4 |
| Toothpaste base (35% by weight sodium | 98.6 |
| bicarbonate) | |
| TOTAL | 100% |

### Example 6

### Example 6A: Flavor Composition

An flavor composition of the present disclosure was made as shown in Table 6-1.

TABLE 6-1

| Flavor Composition | WEIGHT % |
|---|---|
| 1-(2-hydroxy-4-methylcyclohexyl)-ethanone | 14.3% |
| Additional cooling materials | 63.3 |
| Other flavor materials | 22.4 |
| TOTAL | 100% |

### Example 6B: Toothpaste Composition

A silica toothpaste composition of the present disclosure comprising the flavor composition of Example 6A was made as shown in Table 6-2.

TABLE 6-2

| Toothpaste Composition | WEIGHT % |
|---|---|
| Flavor Composition of Example 7A | 1.4% |
| Silica toothpaste base (with 5% sodium chloride) | 98.6% |
| TOTAL | 100% |

### Example 7

### Example 7A: Flavor Composition

An exemplary flavor composition of the present disclosure is shown in Table 7-1.

TABLE 7-1

| Flavor Composition | WEIGHT % |
|---|---|
| 1-(2-hydroxy-4-methylcyclohexyl)-ethanone | 5% |
| (Coolact HK) | |
| 3-L-menthoxypropan-1-2-diol | 0.2% |
| L-menthyl-3-hydroxybutanoate | 0.2% |
| hydroxyethyl-menthyl ether | 0.1% |
| N-(4-methoxyphenyl)-p- | 1% |
| menthanecarboxamide | |
| Additional cooling materials | 33.5% |
| Other flavor materials | 60% |
| TOTAL | 100% |

### Example 7B: Toothpaste Composition

An exemplary baking soda toothpaste composition of the present disclosure comprising the flavor composition of Example 7A is shown in Table 7-2.

TABLE 7-2

| Toothpaste Composition | WEIGHT % |
|---|---|
| Flavor Composition of Example 8A | 1.5% |
| Toothpaste base (25% by weight sodium | 98.5% |
| bicarbonate) | |
| TOTAL | 100% |

### Example 8

### Example 8A: Flavor Composition

An exemplary flavor composition of the present disclosure is shown in Table 8-1.

TABLE 8-1

| Flavor Composition | WEIGHT % |
|---|---|
| 3-L-menthoxypropan-1-2-diol | 10% |
| L-menthyl-3-hydroxybutanoate | 5% |
| hydroxyethyl-menthyl ether | 1% |
| p-methane-3,8-diol | 3% |
| “Non-active” cooling materials | 31% |
| Other flavor materials | 50% |
| TOTAL | 100% |

### Example 8B: Toothpaste Composition

An exemplary baking soda toothpaste composition of the present disclosure comprising the flavor composition of Example 8A is shown in Table 8-2.

TABLE 8-2

| Toothpaste Composition | WEIGHT % |
|---|---|
| Flavor Composition of Example 9A | 1.5% |
| Toothpaste base (25% by weight sodium | 98.5% |
| bicarbonate) | |
| TOTAL | 100% |

### Example 9: Toothpaste Composition

Example 9 provides for a natural whitening toothpaste composition as shown in Table 9.

TABLE 9

| Toothpaste Composition | WEIGHT % |
|---|---|
| Sodium Bicarbonate | 20.00 |
| Humectant(s) | 44.20 |
| Water | 24.85 |
| Whitening Agent(s) | 0.75 |
| Abrasive(s) | 5.00 |
| Detergent(s) | 4.00 |
| Flavor Composition(s) | 1.20 |
| TOTAL | 100% |

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the device, method, and system of the disclosed subject matter without departing from the spirit or scope of the disclosed subject matter. Thus, it is intended that the disclosed subject matter include modifications and variations that are within the scope of the appended claims and their equivalents.

For any patents, patent applications, publications, product descriptions, and protocols are cited throughout this application, the disclosures of all of which are incorporated herein by reference in their entirety for all purposes.

1. An orally administered, imbibed, or ingested consumer product comprising:
(i) at least about 5% by weight of one or more salts; and
(ii) at least one cooling material selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof;
wherein the at least one cooling material is present in an amount effective to reduce or inhibit the salty taste or after-taste ordinarily imparted by the salts to the mouth of an end user.

2. The consumer product of claim 1, wherein the one or more salts is selected from the group consisting of metallic carbonate salts; metallic chloride salts; metallic fluoride salts; metallic phosphate salts; stannous salts; strontium salts; zinc salts; and combinations thereof.

3. The consumer product of claim 1, wherein the one or more salts is selected from the group consisting of disodium phosphate; disodium ethylenediaminetetraacetic acid; monosodium glutamate; potassium bicarbonate; potassium chloride; sodium bicarbonate; sodium acetate; sodium alginate; sodium ascorbate; sodium benzoate; sodium caseinate; sodium chloride; sodium citrate; sodium dehydroacetate; sodium diacetate; sodium erythorbate; sodium hydroxide; sodium lactate; sodium nitrate; sodium nitrite; sodium polyphosphate; sodium propionate; sodium saccharin; sodium stearoyl lactylate; sodium sulfite; tetrasodium pyrophosphate; trisodium phosphate; stannous fluoride; strontium chloride; zinc acetate; zinc chloride; zinc citrate; zinc fluoride; zinc gluconate; zinc lactate; zinc salicylate; zinc sulphate; and combinations thereof.

4. The consumer product of claim 1, wherein the one or more salts is sodium bicarbonate.

5. The consumer product of claim 1, wherein the one or more salts is sodium chloride.

6. The consumer product of claim 1, comprising at least 10% by weight of the one or more salts; at least about 20% by weight of the one or more salts; at least about 40% by weight of the one or more salts; at least about 50% by weight of the one or more salts; or at least about 60% by weight of the one or more salts.

7. The consumer product of claim 1, wherein the consumer product is an oral care composition.

8. An orally administered, imbibed, or ingested consumer product comprising:
(i) at least 5% by weight of at least one salt selected from the group consisting of metallic carbonate salts; metallic chloride salts; metallic fluoride salts; metallic phosphate salts; stannous salts; strontium salts; zinc salts; and combinations thereof, wherein said at least one salt ordinarily imparts a salty taste to the mouth of the end user of said consumer product; and
(ii) at least one cooling material selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof,
wherein said at least one cooling material is present in an amount effective to reduce or inhibit the salty taste of the consumer product ordinarily imparted by the at least one salts.

9. The consumer product of claim 8, wherein the at least one cooling material is present in an amount between about 0.0001% to about 1%, between about 0.0005% and about 1%, between about 0.001% and about 0.3%, between about 0.01% and 2% by weight, between about 0.01% and 1.5% by weight, between about 0.01 and 1% by weight, between about 0.01% and 0.5% by weight, or between about 0.05% and 0.2% by weight of the consumer product.

10. The consumer product of claim 8 or 9, wherein the consumer product is an oral care composition.

11. The consumer product of claim 1, wherein the consumer product is a toothpaste, a rinse, or a tooth powder.

12-33. (canceled)

34. A method of reducing or inhibiting the salty taste and/or aftertaste of an orally administered, imbibed, or ingested consumer product comprising at least about 5% by weight of one or more salt, the method comprising:
adding to the consumer product a cooling material selected from one of at least one cooling material selected from the group consisting of p-menthane-3,8-diol; hydroxyethyl-menthyl ether; 3-L-menthoxypropan-1-2-diol; L-menthyl-3-hydroxybutanoate; 1-(2-hydroxy-4-methylcyclohexyl)-ethanone; N-(4-methoxyphenyl)-p-menthanecarboxamide; and combinations thereof in an amount effective to reduce or inhibit the salty taste or after-taste ordinarily imparted by the one or more salt.

35. The method of claim 34, wherein the one or more salt ordinarily imparts a salty taste to the mouth of the end user of the consumer product.

36. The method of claim 34 or 35, the one or more salt is selected from the group consisting of metallic carbonate salts; metallic chloride salts; metallic fluoride salts; metallic phosphate salts; stannous salts; strontium salts; zinc salts; and combinations thereof.

37. The method of claim 34, wherein the one or more salt is sodium bicarbonate.

38. The method of claim 34, wherein the one or more salt is sodium chloride.

39. The method of claim 34, wherein the one or more salt is present in an amount of at least 10% by weight; at least about 20% by weight; at least about 40% by weight; at least about 50% by weight; or at least about 60% by weight of the consumer product.

40. The method of claim 34, wherein the at least one cooling material is added in an amount between about 0.0001% to about 1%, between about 0.0005% and about 1%, between about 0.001% and about 0.3%, between about 0.01% and 2% by weight, between about 0.01% and 1.5% by weight, between about 0.01 and 1% by weight, between about 0.01% and 0.5% by weight, or between about 0.05% and 0.2% by weight of the consumer product.

41. The method of claim 34, wherein the consumer product is an oral care composition.

42. The method of claim 34, wherein the consumer product is a toothpaste, a rinse, or a tooth powder.
